(19) United States
(12) Patent Application Publication
(10) Pub. No.: US 2025/0035502 A1
(43) Pub. Date: Jan. 30, 2025

(54) ROBOTIC SYSTEMS AND METHODS FOR DETERMINING HYDROCARBON EMISSIONS

(71) Applicant: Saudi Arabian Oil Company, Dhahran (SA)

(72) Inventors: Soliman A. Walaie, Dammam (SA); Khaled Abu-Salem, Dhahran (SA); Khalid A. Al-Binali, Dammam (SA); Rashed F. Alhajri, Al-Nabiyah (SA); Harish Badrinarayanan, Dhahran (SA); Faleh S. Aldawsari, Hofuf (SA)

(21) Appl. No.: 18/785,349

(22) Filed: Jul. 26, 2024

Related U.S. Application Data
(60) Provisional application No. 63/515,715, filed on Jul. 26, 2023.

(51) Int. Cl. G01M 3/04; G01N 33/00; G05D 1/689; G05D 105/80

(52) U.S. Cl. CPC G01M 3/04; G01N 33/0075; G05D 1/689; G05D 2105/89

(57) ABSTRACT

A robotic system includes a mobile platform; a gas payload suite mounted on the platform and including at least one gas emissions detection sensor; a navigation sensors suite mounted on the platform and including at least one navigation sensor; and a control system communicably coupled to the gas payload suite and the navigation sensor suite and configured to perform operations including identifying gas emissions measurements from the at least one gas emissions detection sensor, determining a location of gas emissions based at least in part on the identified gas emissions measurements, and operating a motion controller to move the mobile platform relative to the determined location of the gas emissions.

### CROSS-REFERENCE TO RELATED APPLICATION

This application claims priority under 35 U.S.C. § 119 to U.S. Provisional Patent Application Ser. No. 63/515,715, filed on Jul. 26, 2023, the entire contents of which are incorporated herein by reference.

### TECHNICAL FIELD

The present disclosure describes a robotic system, such as, for example, an autonomous robotic system, for determining emissions in or from a hydrocarbon environment.

### BACKGROUND

Leaks from hydrocarbon facilities, such as leaks of gas or other hydrocarbon vapors or fluids (especially gasses) can increase greenhouse gasses, elevate safety risks as well as cause economic and other problems. Enhancing an accuracy of detection, mapping, localization, and quantification of such gas leaks can help reduce such emissions and also provide economic benefits. Such detection is most useful in real time or frequent surveys to quickly take measures to stop the leaks.

### SUMMARY

In an example implementation, a robotic system includes a mobile platform; a gas payload suite mounted on the platform and including at least one gas emissions detection sensor; a navigation sensors suite mounted on the platform and including at least one navigation sensor; and a control system communicably coupled to the gas payload suite and the navigation sensor suite and configured to perform operations including identifying gas emissions measurements from the at least one gas emissions detection sensor, determining a location of gas emissions based at least in part on the identified gas emissions measurements, and operating a motion controller to move the mobile platform relative to the determined location of the gas emissions.

In an aspect combinable with the example implementation, the mobile platform has at least one wheel, at least one track, or at least one leg.

In another aspect combinable with one, some, or all of the previous aspects, the operation of operating the motion controller includes operating the least one wheel, at least one track, or at least one leg to move the mobile platform.

In another aspect combinable with one, some, or all of the previous aspects, the at least one gas emissions detection sensor includes at least one of: at least one optical gas imaging (OGI) sensor, at least one tunable diode laser absorption spectroscopy (TDLAS) sensor, or at least one multi-gas sniffer sensor.

In another aspect combinable with one, some, or all of the previous aspects, the at least one navigation sensor includes: at least one global navigation satellite system (GNSS) sensor, at least one inertial navigation system (INS) sensor, at least one LiDAR sensor, or at least one optical or IR sensor

Another aspect combinable with one, some, or all of the previous aspects further includes a communication interface module communicably coupled to the control system, wherein the operations further include operating the communication interface module to provide data associated with the identified gas emissions measurements.

In another aspect combinable with one, some, or all of the previous aspects, the data includes at least the location of the gas emissions or a quantity of the gas emissions.

Another aspect combinable with one, some, or all of the previous aspects further includes at least one power source mounted on the mobile platform and electrically coupled to the gas payload suit, the navigation sensors suite, the communications interface module, and the control system.

In another aspect combinable with one, some, or all of the previous aspects, the at least one power source includes at least one rechargeable battery.

In another aspect combinable with one, some, or all of the previous aspects, the operations further include triangulating the location of gas emissions based at least in part on the identified gas emissions measurements.

In another aspect combinable with one, some, or all of the previous aspects, the operation of triangulating the location includes: identifying a first gas emissions measurement from the at least one gas emissions detection sensor, determining a first location of the gas emissions based at least in part on the identified first gas emissions measurements, operating the motion controller to move the mobile platform to the first location of the gas emissions, identifying a second gas emissions measurement from the at least one gas emissions detection sensor at the first location, determining a second location of the gas emissions based at least in part on the identified second gas emissions measurements, and operating the motion controller to move the mobile platform to a second location of the gas emissions different than the first location.

In another aspect combinable with one, some, or all of the previous aspects, the operation of determining the location of gas emissions based at least in part on the identified gas emissions measurements includes: identifying weather or wind data taken at a time associated with the identified gas emissions measurement, and determining the location of gas emissions based on the identified gas emissions measurements and the identified weather or wind data.

In another aspect combinable with one, some, or all of the previous aspects, the operation of operating the motion controller to move the mobile platform relative to the determined location of the gas emissions includes operating the motion controller to autonomously move the mobile platform relative to the determined location of the gas emissions based on data from the at least one navigation sensor.

In another aspect combinable with one, some, or all of the previous aspects, the operation of operating the motion controller to move the mobile platform relative to the determined location of the gas emissions includes operating the motion controller to semi-autonomously move the mobile platform relative to the determined location of the gas emissions based on data from the at least one navigation sensor and data from the communications interface module.

In another aspect combinable with one, some, or all of the previous aspects, the operation of operating the motion controller to move the mobile platform relative to the determined location of the gas emissions includes operating the motion controller to move the mobile platform relative to the determined location of the gas emissions based on a human-generated command delivered to the control system through the communications interface module.

In another example implementation, a method includes initiating a robotic system, the robotic system. The robotic system includes a mobile platform; a gas payload suite mounted on the platform and including at least one gas emissions detection sensor; and a navigation sensors suite mounted on the platform and including at least one navigation sensor. The method includes operating the robotic system to identify gas emissions measurements from the at least one gas emissions detection sensor, operating the robotic system to determine a location of gas emissions based at least in part on the identified gas emissions measurements, and operating a motion controller to move the mobile platform relative to the determined location of the gas emissions.

In an aspect combinable with the example implementation, operating the motion controller to move the mobile platform includes moving the mobile platform with at least one wheel, at least one track, or at least one leg.

In another aspect combinable with one, some, or all of the previous aspects, moving the mobile platform with at least one wheel, at least one track, or at least one leg includes moving the mobile platform with a plurality of wheels.

In another aspect combinable with one, some, or all of the previous aspects, operating the robotic system to identify gas emissions measurements from the at least one gas emissions detection sensor includes identifying gas emissions measurements with at least one of: at least one optical gas imaging (OGI) sensor, at least one tunable diode laser absorption spectroscopy (TDLAS) sensor, or at least one multi-gas sniffer sensor.

In another aspect combinable with one, some, or all of the previous aspects, operating the robotic system to determine the location of gas emissions based at least in part on the identified gas emissions measurements includes determining the location with the at least one navigation sensor that includes at least one global navigation satellite system (GNSS) sensor, at least one inertial navigation system (INS) sensor, at least one LiDAR sensor, or at least one optical or IR sensor.

Another aspect combinable with one, some, or all of the previous aspects includes operating a communication interface module of the robotic system to provide data associated with the identified gas emissions measurements to a control system.

In another aspect combinable with one, some, or all of the previous aspects, the data includes at least the location of the gas emissions or a quantity of the gas emissions.

Another aspect combinable with one, some, or all of the previous aspects includes providing electrical power to the gas payload suit, the navigation sensors suite, the communications interface module, and the control system with at least one power source mounted on the mobile platform.

In another aspect combinable with one, some, or all of the previous aspects, the at least one power source includes at least one rechargeable battery.

Another aspect combinable with one, some, or all of the previous aspects includes operating the robotic system to triangulate the location of gas emissions based at least in part on the identified gas emissions measurements.

In another aspect combinable with one, some, or all of the previous aspects, operating the robotic system to triangulate the location of gas emissions based at least in part on the identified gas emissions measurements includes operating the robotic system to identify a first gas emissions measurement from the at least one gas emissions detection sensor, determine a first location of the gas emissions based at least in part on the identified first gas emissions measurements, operate the motion controller to move the mobile platform to the first location of the gas emissions, identify a second gas emissions measurement from the at least one gas emissions detection sensor at the first location, determine a second location of the gas emissions based at least in part on the identified second gas emissions measurements, and operate the motion controller to move the mobile platform to a second location of the gas emissions different than the first location.

In another aspect combinable with one, some, or all of the previous aspects, operating the robotic system to determine the location of gas emissions based at least in part on the identified gas emissions measurements includes operating the robotic system to identify weather or wind data taken at a time associated with the identified gas emissions measurement, and determine the location of gas emissions based on the identified gas emissions measurements and the identified weather or wind data.

In another aspect combinable with one, some, or all of the previous aspects, operating the motion controller to move the mobile platform relative to the determined location of the gas emissions includes operating the motion controller to autonomously move the mobile platform relative to the determined location of the gas emissions based on data from the at least one navigation sensor.

In another aspect combinable with one, some, or all of the previous aspects, operating the motion controller to move the mobile platform relative to the determined location of the gas emissions includes operating the motion controller to semi-autonomously move the mobile platform relative to the determined location of the gas emissions based on data from the at least one navigation sensor and data from the communications interface module.

In another aspect combinable with one, some, or all of the previous aspects, operating the motion controller to move the mobile platform relative to the determined location of the gas emissions includes operating the motion controller to move the mobile platform relative to the determined location of the gas emissions based on a human-generated command delivered to the control system through the communications interface module.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an illustration of an example implementation of an autonomous robotic system according to the present disclosure.

FIG. 2 is an architecture of example sub-systems of an autonomous robotic system according to the present disclosure.

FIG. 3 is a schematic diagram of an environment in which an autonomous robotic system can operate according to the present disclosure.

FIG. 4 is an example workflow of an autonomous robotic system according to the present disclosure

FIG. 5 is a control system (or controller) for an autonomous robotic system according to the present disclosure.

### DETAILED DESCRIPTION

The present disclosure describes robotic systems and methods for scanning of unwanted or undesirable gas emissions, detecting such emissions, and quantifying such admissions in an autonomous, semi-autonomous, or manual operation. Example implementations of the robotic systems according to the present disclosure can provide for an intelligent mobile robot (for example, legged, wheeled, or tracked) for the autonomous gas emission detection, localization, quantification, and visualization. In some aspects, example implementations of the robotic systems include one or more of: advanced optical gas imaging (OGI), tunable diode laser absorption spectroscopy (TDLAS), light detection and ranging (LiDAR), and/or multi-gas sniffer (Flame Ionization Detectors-FID or Photo Ionization Detectors-PID) to perform autonomous gas/methane emission detection, localization, quantification and visualization of gas emissions on a three-dimensional (3D model or digital twin. In some example implementations, artificial intelligence (AI) and data analytics are applied with the robotic system to triangulate, correlate, and integrate collected data to precisely detect, measure, localize, and trace gas (for example, methane or otherwise) emissions. In some aspects, an AI module of the robotic system can implement algorithms that compile, triangulate, analyze, and diffuse collected data from the different sensors (gas sensors, LiDAR, GNSS), overlay diffused data with optical (RGB) and thermal camera to generate Digital Twin (3D model) and visualize gas leaks locations, quantity, and spread.

The example implementations of the robotic system according to the present disclosure can operate manually, semi-autonomously or fully autonomously across one or more hydrocarbon facilities to detect gas emissions and leaks. The robotic systems according to the present disclosure can enable effective, safe, and efficient gas emissions surveys and assessments through an integration of advanced gas emission sensors and scanning techniques that utilize triangulation, integration and correlation of multiple data feeds (in other words, data payload).

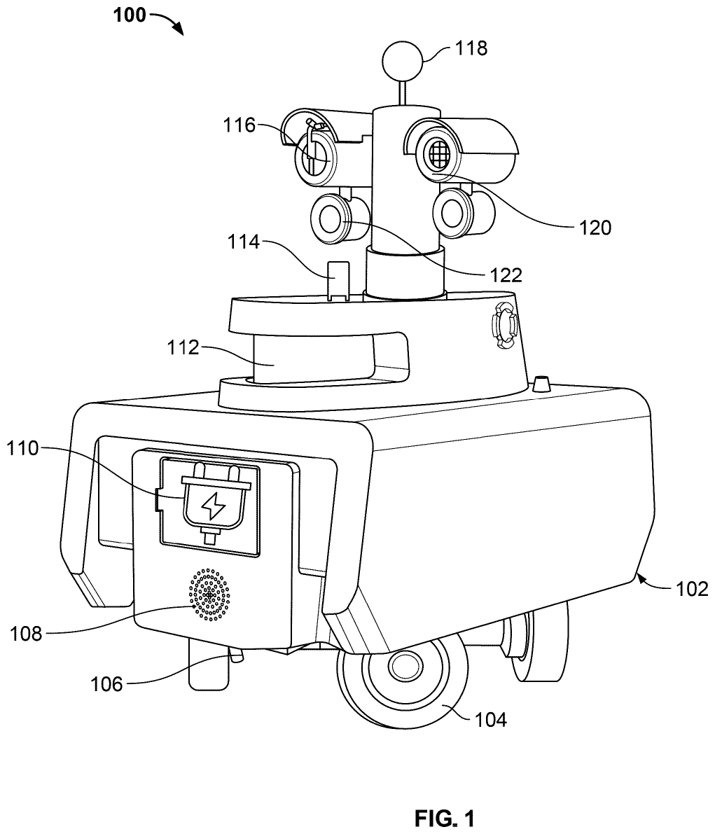

FIG. 1 is an illustration of an example implementation of an autonomous robotic system 100 according to the present disclosure. Although termed an “autonomous” robotic system, system 100 can operate to detect, localize, and quantify emissions leaks (as well as other tasks) in multiple, adjustable modes, including fully autonomous (for example, operating without human intervention in accordance with a pre-specified set of instructions and algorithms), semi-autonomously (for example, operating with some human intervention in combination with a pre-specified set of instructions and algorithms), or manually (for example, operating in accordance with instructions and commands provided by a human operator).

Robotic system 100 includes a platform 102 in which other components and sub-systems are mounted and supported. In this example, wheels 104 are coupled to the platform 102 to allow movement of the robotic system 100 (either autonomously or manually) across a surface, such as ground or man-made surface. However, other forms of movement can also be provided, such as rollers, tracks, or legs, that allows the platform 102 to move during operation of the robotic system 100 for different type terrains and sites conditions.

In this example implementation, the robotic system 100 includes one or more sensor arrays (for example, as part of a gas payload suite) that can operate to, for example, detect or localize (or both) gas emissions. For example, as shown in this example, the robotic system 100 includes a multi-gas sensor (sniffer) 118, an optical gas imaging (OGI) camera 120, and a tunable diode laser absorption spectroscopy (TDLAS) sensor 122 for localized and remote gas sensing. Although each of these sensor arrays are shown, alternative implementations can include fewer or more sensor arrays, as well as multiple sensors in each type of array (for example, multiple sniffers 118, multiple OGI cameras 120, multiple TDLAS sensors 122).

As further shown in this example, the robotic system 100 includes one or more sub-assemblies (for example, as part of a navigation sensors suite that is coupled to a motion controller) that facilitate movement toward a predetermined location or help avoid obstacles. For example, this example of the robotic system 100 includes an optical/IR camera 116, a warning light/ESD 114, a LiDAR system 112, and an obstacle sensor 106. Although each of these sub-assemblies are shown, alternative implementations can include fewer or more sub-assemblies, as well as multiple of each type of sub-assembly (for example, multiple optical/IR cameras 116, multiple warning light/ESD 114, multiple obstacle sensors 106).

As shown in FIG. 1, the robotic system 100 includes a charging adapter 110, which can be a contact charger or contactless charger. For example, in some aspects, the robotic system 100 can includes one or more rechargeable batteries that can provide power to the illustrated components. Each of the batteries can be recharged through the charging adapter 110. A microphone/speaker 108 (for example, as part of a communication module) can also be provided with the robotic system 100, to allow for the robotic system 100 to communicate with human operators, as well as for voice commands to be received by the robotic system 100 from a human operator.

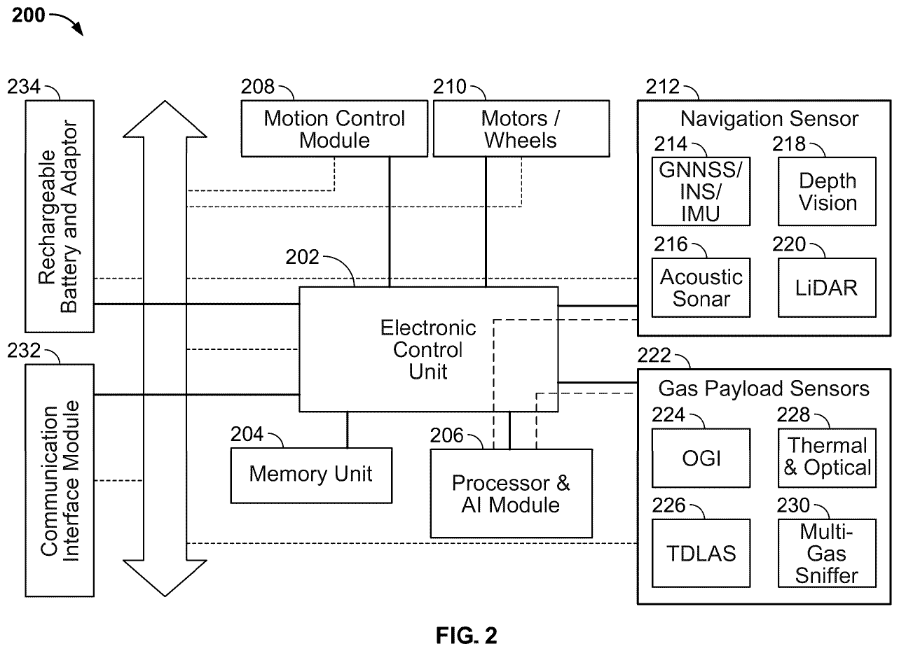

FIG. 2 is an architecture 200 of example sub-systems of the autonomous robotic system 100 according to the present disclosure. In this example, architecture 200 includes a number of sub-systems that provide for the initiation (for example, powering on or otherwise) and operation of the robotic system 100 as it moves, detects, localizes, and reports gas emissions in a hydrocarbon environment. In this example, architecture 200 includes a control system that comprises an electronic control unit (ECU) 202 communicably coupled with a memory unit 204 and a processing/AI module 206. The memory unit 204 can store instructions (for example, in software or firmware) that provide commands carried out by the ECU 202 and/or processing/AI module 206 to control and operate other sub-systems of the architecture 200 (and the robotic system 100, as a whole).

The architecture 200 in this example, as includes a motion control module 208 and a motor/wheels module 210, each of which is communicably coupled to the control system to obtain commands as well as provide feedback thereto. The motion control module 208 and a motor/wheels module 210 can combine to form a motion controller, which manages navigation and motion control operation (autonomous, manual, semi-autonomous), routes, and missions management of the robotic system 100.

The architecture 200 also includes, in this example, a navigation sensors suite (NSS) 212 communicably coupled to the control system. In this example, the NSS 212 includes a global navigation satellite system (GNSS) with inertial navigation system (INS) module 214, a depth vision module 218, an acoustic sonar module 216, and a LiDAR module 220 (for example, LiDAR 112). The NSS 212 operates to provide for accurate and precise autonomous navigation of the robotic system 100 through laser scanning and ranging, depth camera(s) and sonar/acoustic collision avoidance sensors.

The illustrated architecture 200 also include a gas payload suite 222 communicably coupled to the control system, which includes an OGI module 224, one or more thermal/optical sensors 228, a TDLAS module 226, and a multi-gas sniffer module 230. The gas payload suite 222 can operate to detect, localize, quantify, and visualize gas emissions (such as methane). In some aspects, the gas payload suite 222 can perform scanning and sniffing of gases based on commands from the ECU 202 and feed data to the processing/AI module 206 for analysis.

In the architecture 200, the control system operates to analyze data from the gas payload suite 222 to correlate and triangulate such data with information from the NSS 212, including the LiDAR data. Other data can be provided to the control system from sensors on the robotic system 100 or from a feed of data (for example, wireless feed), such as weather conditions and/or wind speed (for instance, taken at or near a time when gas emissions are detected or measured). Based on these sets of data provided, the control system (working with the NSS 212, gas payload suite 222, and motion controller) can operate the robotic system 100 to precisely detect; localize, quantify and visualize leaks on digital maps. For example, the processing/AI module 206 can use the data feeds (analysis of current weather, previously collected gas leak data) to generate 3D maps of emissions detections, as well as report differentials and progressions in emissions detections.

In some examples, the gas payload suite 222 provides for 360° scanning of gas emissions by the robotic system 100. For instance, all or part of the gas payload suite 222 can be mounted on a three axis gimbal (gyro) to provide highly stabilized and rotational gas scanning capability of the robotic system 100.

As further shown in architecture 200, a rechargeable battery and adapter module 234 is provided and is electrically coupled to other sub-systems of the architecture 200 to provide electrical power for operations. For example, as shown, the rechargeable battery and adapter module 234 is electrically coupled to the motion controller, the control systems, the NSS 212, a communications interface module 232, and the gas payload suite 222 to provide electrical power to these components (and their sub-components, if needed).

Architecture 200, in this example, also includes the communication interface module 232, which is coupled to control system (through the ECU 202). In some aspects, the communication interface module 232 operates to provide for onboard and remote communication (for example, wireless), as well as data and telemetry transfer, encryption, and control of the robotic system 100.

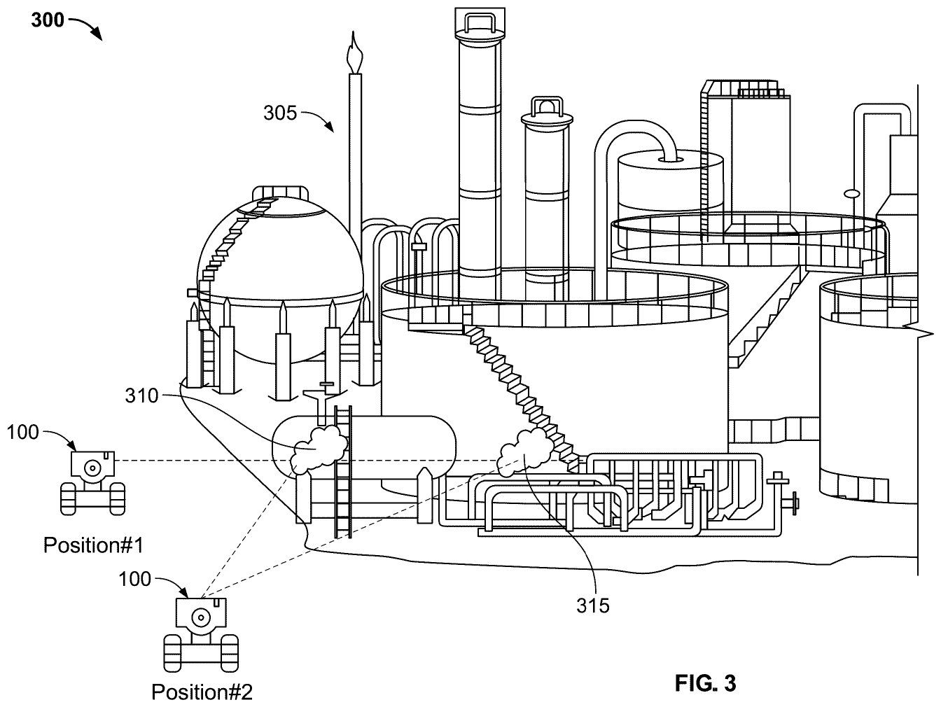

FIG. 3 is a schematic diagram of an environment 300 in which an autonomous robotic system can operate according to the present disclosure. In this example, environment 300 includes a hydrocarbon facility 305. An example of the hydrocarbon facility 305 is a refinery or other hydrocarbon processing facility where produced hydrocarbons are processed and refined into multiple commercial hydrocarbon streams (as shown in this example). However, a hydrocarbon facility can also include a hydrocarbon production site, such as a drilling rig (on or off shore), a hydrocarbon transfer site (where oil or gas trucks or tankers are loaded or off-loaded of hydrocarbons) or any other hydrocarbon site where gas emissions, such as methane emissions, can occur and need to be detected, localized, and/or quantified.

In this example, gas emissions 310 and 315 can occur at different locations in the hydrocarbon facility 305. In FIG. 3, the autonomous robotic system 100 can move from or between positions 1 and 2, as shown, as well as other positions, to detect, triangulate, localize, and quantify the gas emissions 310 and 315. For example, the processing and AI module 206 can receive or identify detected gas emissions from the gas payload suite 222, as well as location data from the NSS 212, to triangulate and localize gas emissions 310 and 315. Other data, such as weather data or wind data provided to the autonomous robotic system 100, for example, through the communication interface module 232, can also be used by the processing and AI module 206. In some aspects, the detection, localization, and triangulation process is an iterative process that involves movement of the autonomous robotic system 100 (through the motion controller and control system in combination with the NSS 212) to re-position at multiple locations as guided by the processing and AI module 206.

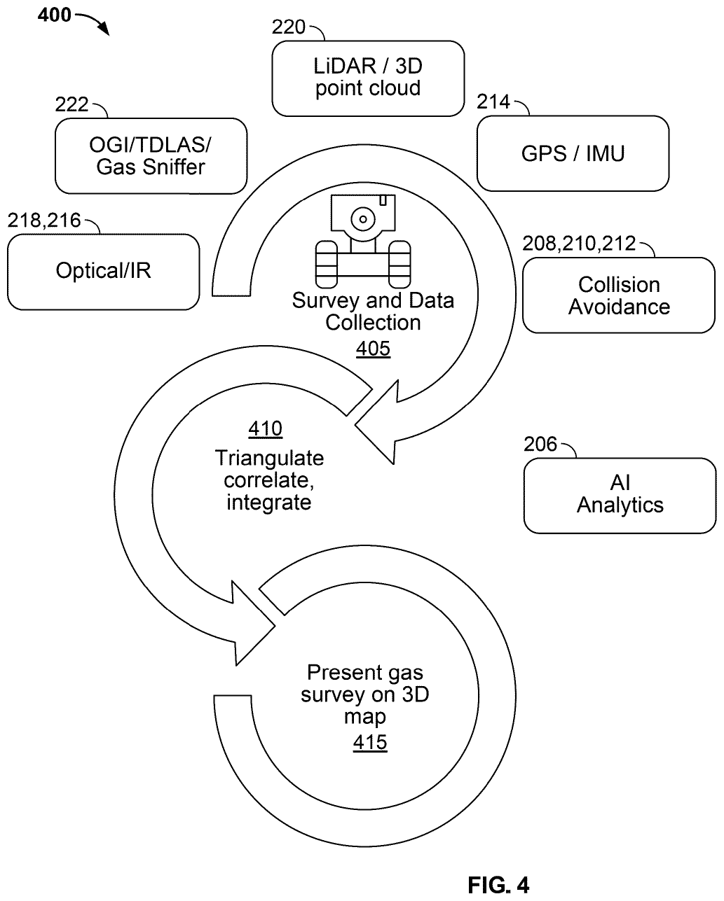

FIG. 4 is an example workflow 400 of an autonomous robotic system according to the present disclosure. For example, implementations of the robotic system 100 (whether autonomous, semi-autonomous, or manual) generally can perform at least three different functions, including: survey and data collection 405; triangulation, correlation, and integration (TCI) 410; and data presentation 415. Workflow 400 shows an example of particular components (from the architecture 200 of the robotic system 100) that operate for each functions shown here.

In an example operation (or set of operations) of an autonomous robotic system according to the present disclosure, robotic system 100 can be activated, powered on, or otherwise initialed to perform operations such as survey and data collection 405; triangulation, correlation, and integration (TCI) 410; and data presentation 415. In some aspects, survey and data collection 405 operations can include, for example, operating the robotic system 100 to identify gas emissions measurements from a gas emissions detection sensor (such as one or more of the multi-gas sensor (sniffer) 118, the OGI camera 120, or the TDLAS sensor 122) or, for instance, the gas payload suite 222.

In some aspects, TCI 410 can include operating the robotic system 100 to determine a location of the detected gas emissions with, for example, the gas payload suite 222 and the NSS 212. The TCI 410 can also include moving the robotic system 100 to the determined location of the gas emissions. In some aspects, for instance, motion control module 208 and motor/wheels module 210 operate, in combination with a control system for example, to move robotic system autonomously, semi-autonomously, or by command of an operator).

Data presentation 415 can include operating the robotic system 100 to analyze and provide visual presentation (for example, to an operator through the robotic system 100 or remotely from the robotic system 100) the gas emissions data, triangulation data, localization data, or otherwise. For example, communication interface module 232 can operate to provide onboard and remote communication, as well as data and telemetry transfer, encryption, and control of the robotic system 100.

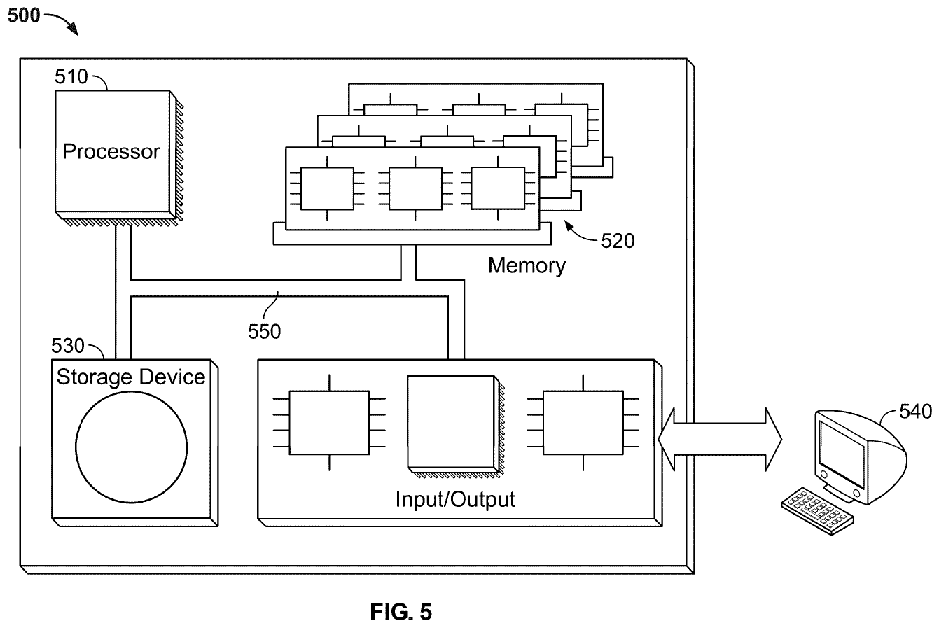

FIG. 5 is an example control system (or controller) 500 for a robotic system, such as an autonomous robotic system as shown in FIG. 1 according to the present disclosure. For example, all or parts of the controller 500 can be used for the operations described previously, for example as or as part of the control system described with reference to the architecture 200 of FIG. 2. The controller 500 is intended to include various forms of digital computers, such as printed circuit boards (PCB), processors, digital circuitry, or otherwise. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 500 includes a processor 510, a memory 520, a storage device 530, and an input/output device 540. Each of the components 510, 520, 530, and 540 are interconnected using a system bus 550. The processor 510 is capable of processing instructions for execution within the controller 500. The processor may be designed using any of a number of architectures. For example, the processor 510 may be a CISC (Complex Instruction Set Computers) processor, a RISC (Reduced Instruction Set Computer) processor, or a MISC (Minimal Instruction Set Computer) processor.

In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the controller 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the controller 500. In one implementation, the storage device 530 is a computer-readable medium. In various different implementations, the storage device 530 may be a floppy disk device, a hard disk device, an optical disk device, a tape device, flash memory, a solid state device (SSD), or a combination thereof.

The input/output device 540 provides input/output operations for the controller 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, solid state drives (SSDs), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) or LED (light-emitting diode) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include wireless and/or wired local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

What is claimed is:

1. A robotic system, comprising:
a mobile platform;
a gas payload suite mounted on the platform and comprising at least one gas emissions detection sensor;
a navigation sensors suite mounted on the platform and comprising at least one navigation sensor; and
a control system communicably coupled to the gas payload suite and the navigation sensor suite and configured to perform operations comprising:
identifying gas emissions measurements from the at least one gas emissions detection sensor;
determining a location of gas emissions based at least in part on the identified gas emissions measurements; and
operating a motion controller to move the mobile platform relative to the determined location of the gas emissions.

2. The robotic system of claim 1, wherein the mobile platform has at least one wheel, at least one track, or at least one leg.

3. The robotic system of claim 2, wherein the operation of operating the motion controller comprises operating the least one wheel, at least one track, or at least one leg to move the mobile platform.

4. The robotic system of claim 1, wherein the at least one gas emissions detection sensor comprises at least one of:
at least one optical gas imaging (OGI) sensor,
at least one tunable diode laser absorption spectroscopy (TDLAS) sensor, or
at least one multi-gas sniffer sensor.

5. The robotic system of claim 1, wherein the at least one navigation sensor comprises:
at least one global navigation satellite system (GNSS) sensor,
at least one inertial navigation system (INS) sensor,
at least one LiDAR sensor, or
at least one optical or IR sensor.

6. The robotic system of claim 1, further comprising a communication interface module communicably coupled to the control system, wherein the operations further comprise operating the communication interface module to provide data associated with the identified gas emissions measurements.

7. The robotic system of claim 6, wherein the data comprises at least the location of the gas emissions or a quantity of the gas emissions.

8. The robotic system of claim 1, further comprising at least one power source mounted on the mobile platform and electrically coupled to the gas payload suit, the navigation sensors suite, the communications interface module, and the control system.

9. The robotic system of claim 8, wherein the at least one power source comprises at least one rechargeable battery.

10. The robotic system of claim 1, wherein the operations further comprise triangulating the location of gas emissions based at least in part on the identified gas emissions measurements.

11. The robotic system of claim 10, wherein the operation of triangulating the location comprises:
identifying a first gas emissions measurement from the at least one gas emissions detection sensor;
determining a first location of the gas emissions based at least in part on the identified first gas emissions measurements;
operating the motion controller to move the mobile platform to the first location of the gas emissions;
identifying a second gas emissions measurement from the at least one gas emissions detection sensor at the first location;
determining a second location of the gas emissions based at least in part on the identified second gas emissions measurements; and
operating the motion controller to move the mobile platform to a second location of the gas emissions different than the first location.

12. The robotic system of claim 1, wherein the operation of determining the location of gas emissions based at least in part on the identified gas emissions measurements comprises:
identifying weather or wind data taken at a time associated with the identified gas emissions measurement; and
determining the location of gas emissions based on the identified gas emissions measurements and the identified weather or wind data.

13. The robotic system of claim 1, wherein the operation of operating the motion controller to move the mobile platform relative to the determined location of the gas emissions comprises operating the motion controller to autonomously move the mobile platform relative to the determined location of the gas emissions based on data from the at least one navigation sensor.

14. The robotic system of claim 1, wherein the operation of operating the motion controller to move the mobile platform relative to the determined location of the gas emissions comprises operating the motion controller to semi-autonomously move the mobile platform relative to the determined location of the gas emissions based on data from the at least one navigation sensor and data from the communications interface module.

15. The robotic system of claim 1, wherein the operation of operating the motion controller to move the mobile platform relative to the determined location of the gas emissions comprises operating the motion controller to move the mobile platform relative to the determined location of the gas emissions based on a human-generated command delivered to the control system through the communications interface module.

16. A method, comprising:
initiating a robotic system, the robotic system comprising:
a mobile platform;
a gas payload suite mounted on the platform and comprising at least one gas emissions detection sensor; and
a navigation sensors suite mounted on the platform and comprising at least one navigation sensor;
operating the robotic system to identify gas emissions measurements from the at least one gas emissions detection sensor;
operating the robotic system to determine a location of gas emissions based at least in part on the identified gas emissions measurements; and
operating a motion controller to move the mobile platform relative to the determined location of the gas emissions.

17. The method of claim 16, wherein operating the motion controller to move the mobile platform comprises moving the mobile platform with at least one wheel, at least one track, or at least one leg.

18. The method of claim 17, wherein moving the mobile platform with at least one wheel, at least one track, or at least one leg comprises moving the mobile platform with a plurality of wheels.

19. The method of claim 16, wherein operating the robotic system to identify gas emissions measurements from the at least one gas emissions detection sensor comprises identifying gas emissions measurements with at least one of:
at least one optical gas imaging (OGI) sensor,
at least one tunable diode laser absorption spectroscopy (TDLAS) sensor, or
at least one multi-gas sniffer sensor.

20. The method of claim 16, wherein operating the robotic system to determine the location of gas emissions based at least in part on the identified gas emissions measurements comprises determining the location with the at least one navigation sensor that comprises:
at least one global navigation satellite system (GNSS) sensor,
at least one inertial navigation system (INS) sensor,
at least one LiDAR sensor, or
at least one optical or IR sensor.

21. The method of claim 16, comprising operating a communication interface module of the robotic system to provide data associated with the identified gas emissions measurements to a control system.

22. The method of claim 21, wherein the data comprises at least the location of the gas emissions or a quantity of the gas emissions.

23. The method of claim 21, comprising providing electrical power to the gas payload suit, the navigation sensors suite, the communications interface module, and the control system with at least one power source mounted on the mobile platform.

24. The method of claim 23, wherein the at least one power source comprises at least one rechargeable battery.

25. The method of claim 16, comprising operating the robotic system to triangulate the location of gas emissions based at least in part on the identified gas emissions measurements.

26. The method of claim 25, wherein operating the robotic system to triangulate the location of gas emissions based at least in part on the identified gas emissions measurements comprises operating the robotic system to:
identify a first gas emissions measurement from the at least one gas emissions detection sensor;
determine a first location of the gas emissions based at least in part on the identified first gas emissions measurements;
operate the motion controller to move the mobile platform to the first location of the gas emissions;
identify a second gas emissions measurement from the at least one gas emissions detection sensor at the first location;
determine a second location of the gas emissions based at least in part on the identified second gas emissions measurements; and
operate the motion controller to move the mobile platform to a second location of the gas emissions different than the first location.

27. The method of claim 16, wherein operating the robotic system to determine the location of gas emissions based at least in part on the identified gas emissions measurements comprises operating the robotic system to:
identify weather or wind data taken at a time associated with the identified gas emissions measurement; and
determine the location of gas emissions based on the identified gas emissions measurements and the identified weather or wind data.

28. The method of claim 16, wherein operating the motion controller to move the mobile platform relative to the determined location of the gas emissions comprises operating the motion controller to autonomously move the mobile platform relative to the determined location of the gas emissions based on data from the at least one navigation sensor.

29. The method of claim 16, wherein operating the motion controller to move the mobile platform relative to the determined location of the gas emissions comprises operating the motion controller to semi-autonomously move the mobile platform relative to the determined location of the gas emissions based on data from the at least one navigation sensor and data from the communications interface module.

30. The method of claim 16, wherein operating the motion controller to move the mobile platform relative to the determined location of the gas emissions comprises operating the motion controller to move the mobile platform relative to the determined location of the gas emissions based on a human-generated command delivered to the control system through the communications interface module.
